(19) United States
(12) Patent Application Publication
(10) Pub. No.: US 2024/0058686 A1
(43) Pub. Date: Feb. 22, 2024

(54) SMART WEARABLE DEVICE

(71) Applicant: SURGE SPORTSTECH, INC., Redwood City, CA (US)

(72) Inventors: Sandeep Shridhar BHANDARKAR, Mumbai (IN); Krishna RAMANATHAN, Mumbai (IN)

(73) Assignee: SURGE SPORTSTECH, INC., Redwood City, CA (US)

(21) Appl. No.: 18/235,342

(22) Filed: Aug. 17, 2023

(30) Foreign Application Priority Data
Aug. 19, 2022 (IN) 202211047277

(51) Int. Cl. A63B 71/06

(52) U.S. Cl. CPC A63B 71/0622; A63B 2220/803; A63B 2220/836; A63B 2225/20; A63B 2225/50; A63B 2230/00

(57) ABSTRACT

A smart wearable device may include a smart ring device. The smart ring device may include a ring that includes a coupling mount. The smart ring device may also include an electronics unit selectively attachable to the coupling mount. The electronics unit may generate usage data representative of a movement and a physiological response of a user. In addition, the smart ring device may include a feedback system communicatively coupled to the electronics unit. The feedback system may display information related to the smart ring device.

### FIELD

The embodiments discussed in the present disclosure are related to a smart wearable device.

### BACKGROUND

Unless otherwise indicated in the present disclosure, the materials described in the present disclosure are not prior art to the claims in the present application and are not admitted to be prior art by inclusion in this section.

Exercise is a beneficial activity that may improve physical and mental health of a user, among other benefits. Exercising may include aerobic exercises, plyometric exercises, and/or other types of exercise directed at improving the health of the user. Sensors and other electronics may measure exercise/movement related metrics during exercise.

The subject matter claimed in the present disclosure is not limited to embodiments that solve any disadvantages or that operate only in environments such as those described above. Rather, this background is only provided to illustrate one example technology area where some embodiments described in the present disclosure may be practiced.

### SUMMARY

This Summary is provided to introduce a selection of concepts in a simplified form that are further described below in the Detailed Description. This Summary is not intended to identify key features or essential characteristics of the claimed subject matter, nor is it intended to be used as an aid in determining the scope of the claimed subject matter.

A smart wearable device may include a smart ring device. The smart ring device may include a ring that includes a coupling mount. The smart ring device may also include an electronics unit selectively attachable to the coupling mount. The electronics unit may generate usage data representative of a movement and a physiological response of a user. In addition, the smart ring device may include a feedback system communicatively coupled to the electronics unit. The feedback system may display information related to the smart ring device.

### BRIEF DESCRIPTION OF DRAWINGS

Example embodiments will be described and explained with additional specificity and detail through the use of the accompanying drawings in which:

FIG. 1A illustrates a front, a top, and a side view of a smart ring device;

FIG. 1B illustrates isometric views of the smart ring device including various sensors;

FIG. 1C illustrates an exploded view of the smart ring device;

FIG. 1D illustrates additional isometric views of the smart ring device;

FIG. 1E illustrates isometric views of the smart ring device including a coupling mount for the smart ring device and an electronics enclosure;

FIG. 1F illustrates different views of the electronics enclosure including a shape and location of a magnetic charging port;

FIG. 2 illustrates the smart ring device on a finger of a user;

FIG. 3 illustrates a smart belt device including an electronics enclosure, and

FIG. 4 illustrates a front, a perspective, a side, and a top view of a smart clip device, all according to at least one embodiment described in the present disclosure.

### DETAILED DESCRIPTION

Exercise is a beneficial activity that may improve physical and mental health of a user, among other benefits. Exercising may include aerobic exercises, plyometric exercises, and/or other types of exercise directed at improving health of the user. Sensors and other electronics may measure exercise/movement related metrics during exercise.

The use of sensors and electronics to measure exercise/movement related metrics may allow for a watchful feedback loop to monitor an otherwise untrained user. That combined with sensors to monitor physiological response to the exercise may provide a holistic feedback system that allows for the user to optimize training, nutrition, recovery or some combination thereof. Sensors may be used to generate usage data related to the exercise, a medical rehabilitation, a physical rehabilitation, a senior motion tracking, or some combination thereof of the user.

Generating the usage data related to movement of different parts of a body of the user (e.g., different appendages, a left arm, a right arm, a left leg, a right leg, an upper body, a lower body, etc.) may be difficult using a sensor positioned at a single position on the body. Some sensor technologies may generate the usage data representative of movement of different parts of the body of the user using a plethora of sensors and/or data collection products that are paired to a remote device (e.g., a smart watch, a smart phone, etc.). Other sensor technologies may generate the usage data of the different parts of the body of the user using multiple independent devices (e.g., manufactured, sold, produced, etc. by different entities) that interface with different applications.

These sensor technologies may require multiple devices to be charged and used. These sensor technologies may also be attached to the body of the user at positions that are obtrusive, inconvenient, or some combination thereof to the user. In addition, these sensor technologies may not permit or may create problems trying to cross reference the usage data. For example, a first sensor may provide heart rate data to a first application and a second sensor may provide cadence data to a second application that is not compatible with the first application. Further, these sensor technologies may cause the user to navigate, manage, or some combination thereof multiple different applications, web pages, or some combination thereof to view the usage data for a single workout. These sensor technologies may not be designed to be used for different types of exercise equipment.

Therefore, a smart wearable device (e.g., an integrated device) that generates the usage data tracking movement, physiological states, wellness, or some combination thereof of the user while including a minimal footprint and is ideally positioned relative the body of the user may be implemented. In addition, the smart wearable device may permit the user to collect, view, and analyze the usage data in a single application.

Some embodiments described in the present disclosure may include a smart wearable device that generates the usage data representative of movement and/or a physiological state of the user in a minimal and unobtrusive manner. The smart wearable device may include multiple smart components (e.g., two smart ring devices and a central smart device or two smart rings) that generate the usage data representative of movement of and/or the physiological state of the user during the exercise. The smart wearable device may permit measurement and detection of bilateral movement (e.g., movement of the left arm and the right arm, the left leg and the right leg, the upper body and the lower body, etc.) during the exercise.

The smart ring devices may include multiple sensors, processor units, or some combination thereof. The smart ring devices may also include communication systems, feedback systems, or some combination thereof. The sensors, the processor units, the communication systems, the feedback systems, or some combination thereof may be housed/enclosed within a minimal footprint of a ring. The smart ring devices may generate and/or collect the usage data pertaining to the movement and physiological states (e.g., physiological response) of the user during exercise to provide meaningful feedback and analytics to the user.

These and other embodiments of the present disclosure will be explained with reference to the accompanying figures. It is to be understood that the figures are diagrammatic and schematic representations of such example embodiments, and are not limiting, nor are they necessarily drawn to scale. In the figures, features with like numbers indicate like structure and function unless described otherwise.

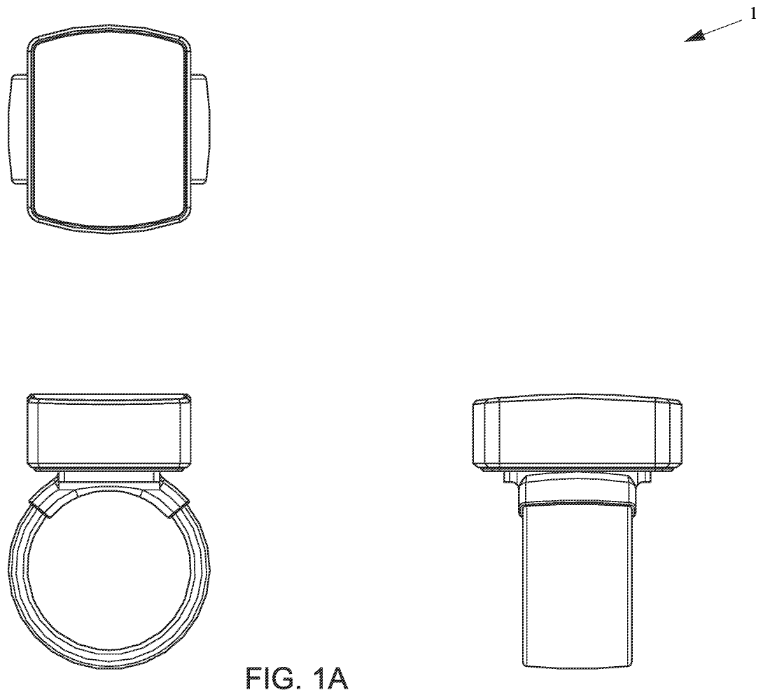

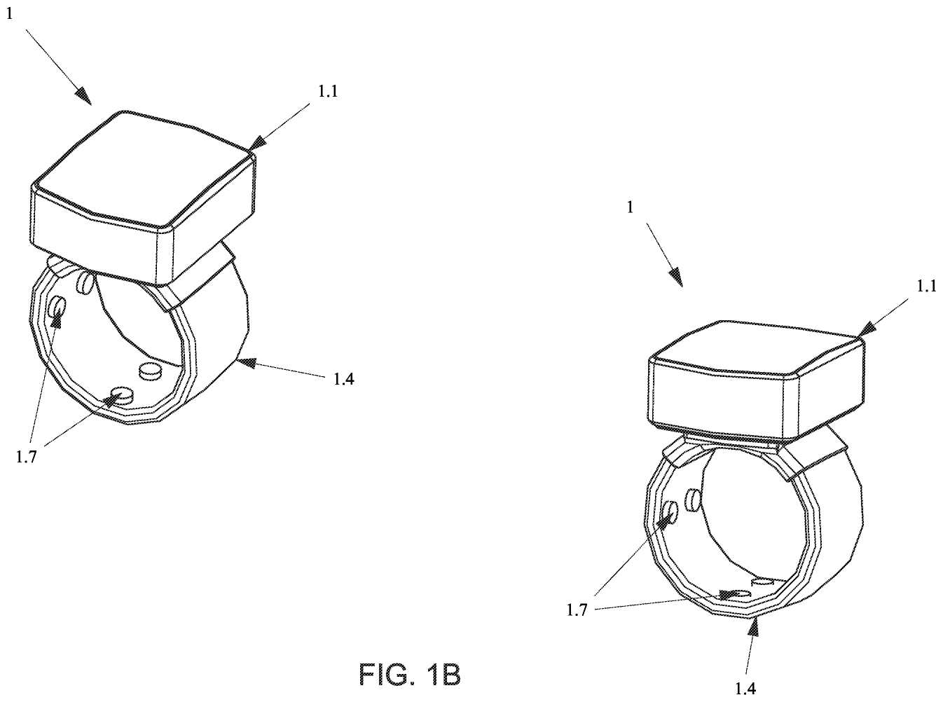

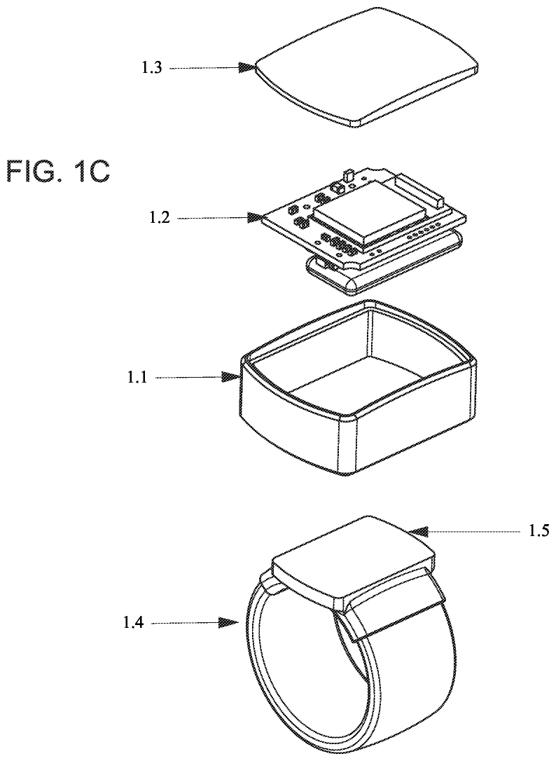

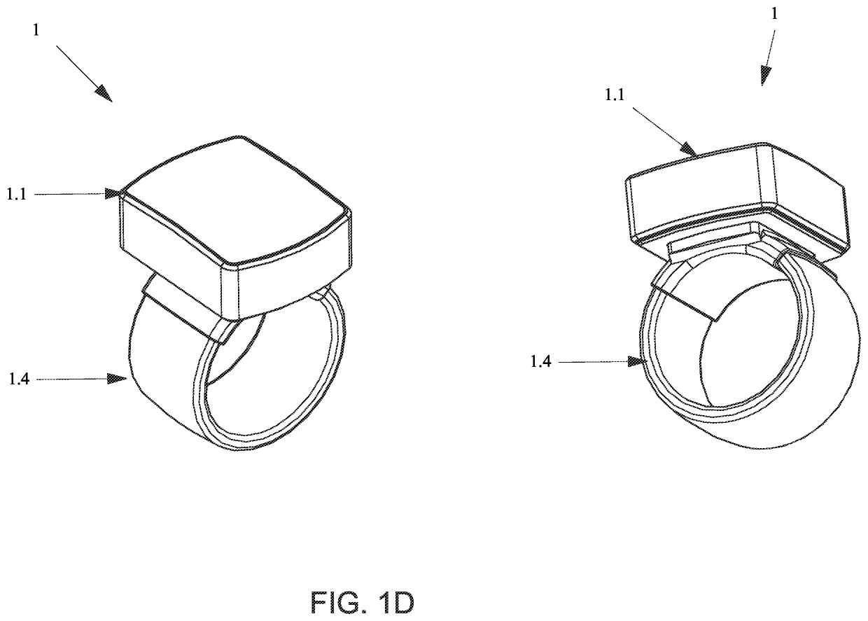

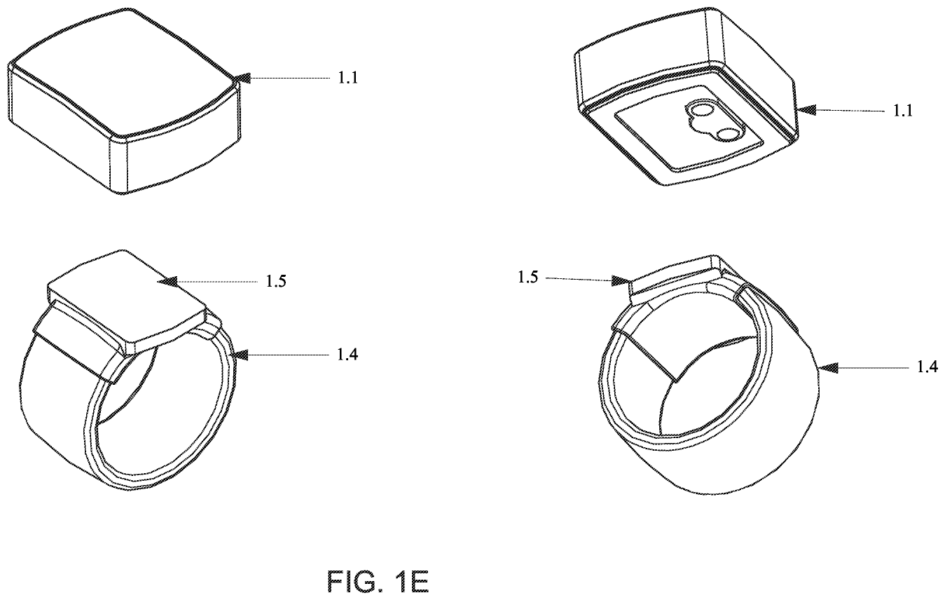

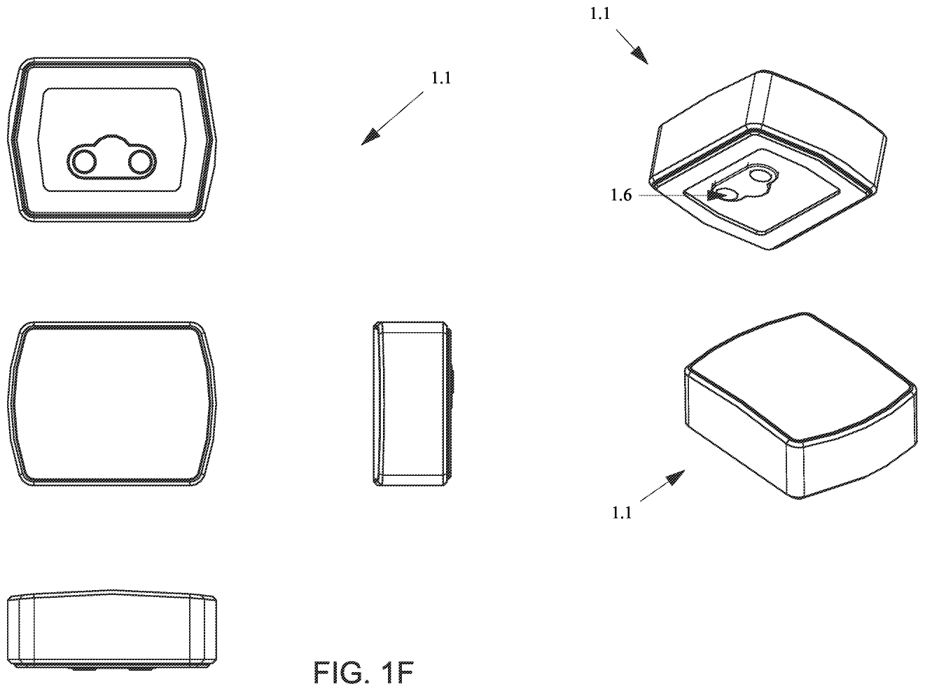

FIG. 1A illustrates a front, a top, and a side view of a smart ring device 1, in accordance with at least one embodiment of the present disclosure. FIG. 1B illustrates isometric views of the smart ring device 1 including various sensors, in accordance with at least one embodiment of the present disclosure. FIG. 1C illustrates an exploded view of the smart ring device 1, in accordance with at least one embodiment of the present disclosure. FIG. 1D illustrates additional isometric views of the smart ring device 1, in accordance with at least one embodiment of the present disclosure. FIG. 1E illustrates isometric views of the smart ring device 1 including a coupling mount 1.5 for the smart ring device 1 and an electronics enclosure 1.1, in accordance with at least one embodiment of the present disclosure.

An example in which multiples of the smart ring device 1 forms at least part of the smart wearable device will now be discussed. With combined reference to FIGS. 1A-1E, the smart ring devices 1 may include the electronics enclosures 1.1, electronics units 1.2, feedback systems 1.3, rings 1.4, the coupling mounts 1.5, or some combination thereof. The electronics units 1.2 may include motion tracking sensors (e.g., inertial measurement units (IMU)) 1.7, biosensors 1.7, physiological sensors 1.7, or some combination thereof. The motion tracking sensors 1.7 may include accelerometers, gyroscopes, magnetometers, or some combination thereof. The biosensors 1.7 may include optical heart-rate sensors, optical SpO2 sensors, blood pressure sensors, surface electromyography (EMG) sensors, continuous glucose sensors, blood lactate sensors, body temperature sensors, or some combination thereof.

The smart ring devices 1 may include the rings 1.4 so as to be ideally placed for tracking both upper and lower body movements of the user whilst being unobstructive and easy to wear and remove. For example, a first smart ring device 1 may be attached to an arm of the user and a second smart ring device 1 may be attached to a leg of the user. The smart ring devices 1 may include a minimal footprint and a low enough weight to increase a likelihood of the user wearing the smart ring devices 1 and for natural unaltered movement and greater ability to focus on a task.

The motion tracking sensors 1.7 may generate the usage data representative of an acceleration, a velocity, a rest period, a direction of travel, other movements measurements, or some combination thereof of a body part of the user. For example, the motion tracking sensors 1.7 may generate the usage data representative of the acceleration of an arm of the user.

The biosensors 1.7 may generate the usage data representative of the physiological response to the exercise and/or the movement of the user. The biosensors 1.7 may generate the usage data to be representative of a sleep quality, a resting heart rate, an average heart rate, a blood pressure, a blood oxygen percentage, a blood glucose level, a blood lactate value, or some combination thereof of the user. The biosensors 1.7 may generate the usage data for lifestyle related measurements or as a response to the exercise by the user. The biosensors 1.7 may continuously generate the usage data (e.g., collect physiological data) over a period of time (e.g., through a day). Alternatively or additionally, the biosensors 1.7 may be selectively turned on to generate the usage data during particular periods of time (e.g., certain times of day, certain activities, or some combination thereof).

In some embodiments, the surface EMG sensors may permit the user to provide input using gestures (e.g., hand signals, appendage movements, etc.).

The biosensors 1.7 may use photoplethysmography (PPG) to generate the usage data representative of the blood oxygen percentage. The biosensors 1.7 may provide light to an appendage of the user and may measure an amount of the light that is reflected by the appendage and/or reflected by blood vessels in living tissue of the appendage of the user to generate the usage data. For example, the biosensors 1.7 may detect a change of blood volume by the PPG technique and to record a volume of blood in a sensor coverage area.

The biosensors 1.7 may include light emitting diode (LED) sensors, photodiode sensors, or some combination thereof. The photodiode sensors may permit the biosensors 1.7 to generate the usage data representative of changes in skin temperature of the user. The LED sensors may permit the biosensors 1.7 to generate the usage data representative of a speed of blood flow and a relative reflection to measure the heart rate, the blood pressure, the oxygen saturation, or some combination thereof of the user. The biosensors 1.7 may generate a red light and a green light and the biosensors 1.7 may detect an amount of the red light and the green light that is received after being proj ected onto the skin of the user (e.g., may obtain a PPG signal) due to reflectivity, penetration, or some combination thereof of the red light and the green light due to the red light penetrating human tissue differently than the green light.

The electronics units 1.2 may include communication systems (e.g., communication modules). The communication systems may include any device, system, component, or collection of components configured to allow or facilitate communication between the electronics units 1.2, the central smart device, a remote device, or some combination thereof. For example, the communication systems may include, without limitation, a modem, a network card (wireless or wired), an infrared communication device, an optical communication device, a wireless communication device (such as an antenna), and/or chipset (such as a Bluetooth device which may include Bluetooth Low Energy, a Near-Field Communication device, an 802.6 device (e.g. Metropolitan Area Network (MAN)), a Wi-Fi device, a WiMAX device, cellular communication facilities, etc.), and/or the like. The communication systems may permit data to be exchanged with any network such as a cellular network, a Wi-Fi network, a MAN, an optical network, etc., to name a few examples, and/or any other devices which may include devices remote from the electronic units. In some embodiments, the remote device may include a personal computing device, such as a mobile phone, mobile computer, tablet computer, and/or similar computing devices.

The communication systems may permit the electronics units 1.2 to transfer the usage data to the remote device. Additionally or alternatively, the communication systems may permit the electronics units 1.2 to transfer processed usage data to the remote device.

In some embodiments, the electronics units 1.2 may include one or more processing units. The processing units may execute instructions stored on any applicable computer-readable storage media. For example, the processing units may include a microprocessor, a microcontroller, a digital signal processor (DSP), an application-specific integrated circuit (ASIC), a Field-Programmable Gate Array (FPGA), or any other digital or analog circuitry configured to interpret and/or to execute program instructions and/or to process data.

The electronics units 1.2 may generate the usage data associated with the use of the device (e.g., data from the motion tracking sensors, the biosensors, or some combination thereof) by the user. The processing units may process the usage data to make determinations regarding the use of an exercise device by the user. Alternatively or additionally, the communication systems may transmit the usage data to the remote device. The remote device may process the usage data to make the determinations regarding the use of the exercise device by the user. For example, after obtaining the usage data, the communication systems may transmit the usage data to a mobile device (e.g., a mobile phone, a tablet computer, a personal computer, etc.), a cloud-based network system, or some combination thereof. In some embodiments, the processed usage data may include information related to a recommended weight for the exercise device (e.g., that the user may benefit from a lighter or a heavier weight), speeding up or slowing down a rate at which a repetition using the exercise device is performed, handling the weight of the exercise device in a different orientation during use, etc.

In some embodiments, the communication systems may transmit the processed usage data to the user. For example, the communication systems may transmit the processed usage data to a user device. The user device may display the processed usage data via a graphical user interface. The user device may include a mobile phone, a personal computer, a tablet computer, etc. In some embodiments, the user device may obtain the processed usage data from the smart ring devices 1, the processing units, the communication systems, the remote device, or some combination thereof. For example, the cloud-based network may process the usage data and the user device may obtain the processed usage data from the cloud-based network.

In some embodiments, the communication systems may transmit the processed usage data to the central smart device. The central smart device may include a mobile phone, a tablet computer, a third smart ring device, etc. In some embodiments, the central smart device may obtain the processed usage data from the smart ring devices, the processing units, the communication systems, the remote device, or some combination thereof.

The feedback systems 1.3 may include the displays, haptic sensors, LEDs, or some combination thereof. The displays may include organic LED (OLED) screens, liquid crystal display (LCD) screens, LED screens, E-ink screens, thin film transistor (TFT)-LCD screens, or any other appropriate display. In some embodiments, the displays may be curved. The displays may display other information such as but not limited to a battery level of the smart ring devices 1, the heart rate, the blood pressure, the blood glucose value, notifications (e.g., smartphone notifications), notifications and alerts related to firmware versions of various connected electronics, a product sub-category, or some combination thereof.

The haptic feedback sensors may provide notifications to the user related to the exercise of the user. For example, the haptic feedback sensors may notify the user that they have completed the exercise, have a certain number of repetitions left, have a certain amount of time remaining, proper or improper form of the user during the exercise, or any other appropriate type of feedback. The displays may display the usage data such as the skin temperature, the oxygen saturation, the heart rate, the acceleration, the velocity, the rest period, the direction of travel, or any other usage data.

In some embodiments the smart ring devices 1 may include both hardware and firmware that permits the smart ring devices 1 to be used alongside a computer application. The smart ring devices 1 may include infrared LEDs to permit the computer application to track the electronics units 1.2 to provide additional workout information to the user. The use of the computer application along with the motion usage data may permit complex movement analysis and may provide usage data for workouts that are a combination of multiple compound movements.

The smart ring devices 1 may include multi-material assemblies that securely house the sensors 1.7 along with efficient mounting of the smart ring devices 1 onto appendages of the user (e.g., fingers).

The electronics enclosures 1.1 may include metal materials, plastic materials, or multi-material enclosure units that house the sensors 1.7, the processor units, the communication systems, the feedback systems, or some combination thereof. The electronics enclosures 1.1 may shield the sensors 1.7, the processor units, the communication systems, the feedback systems, or some combination thereof from dust, water, mechanical shock, debris, or some combination thereof.

The coupling mounts 1.5 may include metal materials, plastic materials, or multi-material components that permit the electronics enclosures 1.1 to be attached to the rings 1.4. The coupling mounts 1.5 may securely fasten the electronics enclosures 1.1 to the rings 1.4. The coupling mounts 1.5 may include bayonets, linear slides with locks, or any other attachment mechanism.

The electronics enclosures 1.1 and the coupling mounts 1.5 may be combined with other clips and clasps so as to fasten the electronics enclosures 1.1 to other positions on the body of the user. Locations that the electronics enclosures 1.1 may be attached to may include a back of a waist, a sternum position, a forearm, a shoulder, between a scapula, a knee, above an ankle, a shoe, etc. The electronics enclosures 1.1 may be transitioned between the different positions. For example, the electronics enclosures 1.1 may be attached to the shoe of the user and the electronics units 1.2 may capture movement data relative the foot, the lower leg, or some combination thereof.

The rings 1.4 may include self-adjusting rings of varying sizing so as to fit a wide range of users. The rings 1.4 may include rigid materials, semi rigid materials, or some combination thereof. The materials of the rings 1.4 may include metal materials, plastic materials, fabric materials, elastomer materials, or some combination thereof to make the rings 1.4 size conforming. The rings 1.4 may provide a secure fit on the appendages of the user. In some embodiments, the rings 1.4 may be one of multiple differently sized rings. Alternatively, the rings 1.4 may include the adjustable ring to fit fingers of different sizes.

FIG. IF illustrates different views of the electronics enclosure 1.1 including a shape and location of a magnetic charging port 1.6, in accordance with at least one embodiment of the present disclosure. In some embodiments, the electronics units 1.2 may include batteries that provide power to the electronics units 1.2. In some embodiments, the batteries may be selectively removable from the smart ring devices 1, the electronics units 1.2, or some combination thereof. The batteries may include rechargeable batteries. In some embodiments, the electronics units 1.2 may recharge the batteries using quick charge devices. Alternatively, or additionally, the batteries may be recharged using wireless charging or magnetic charging. Alternatively, or additionally, the batteries may be recharged using kinetic energy due to movement of the user.

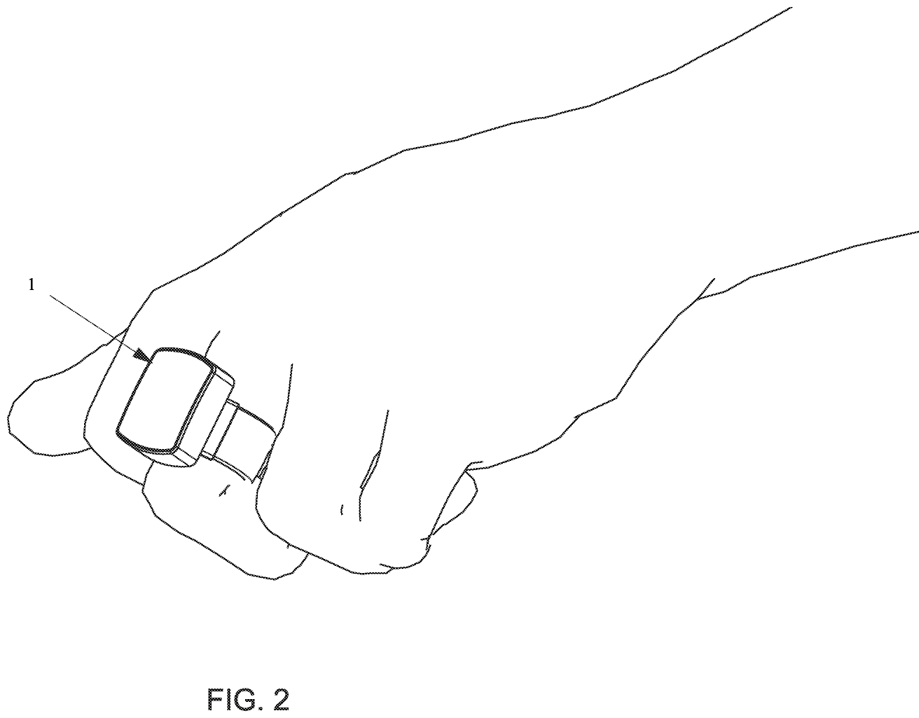

FIG. 2 illustrates the smart ring device 1 on a finger of the user, in accordance with at least one embodiment of the present disclosure. The smart ring devices 1 may attach to one or more fingers of the user.

The smart ring device 1 illustrated in FIGS. 1A-2 as representations of exemplary embodiments of the smart ring device 1 and are not limiting. Modifications, additions, or omissions may be made to the smart ring device 1 without departing from the scope of the present disclosure. For example, in some embodiments, the smart ring device 1 may include the electronics units as an integrated (e.g., embedded) component of the rings 1.4 to form a lower profile.

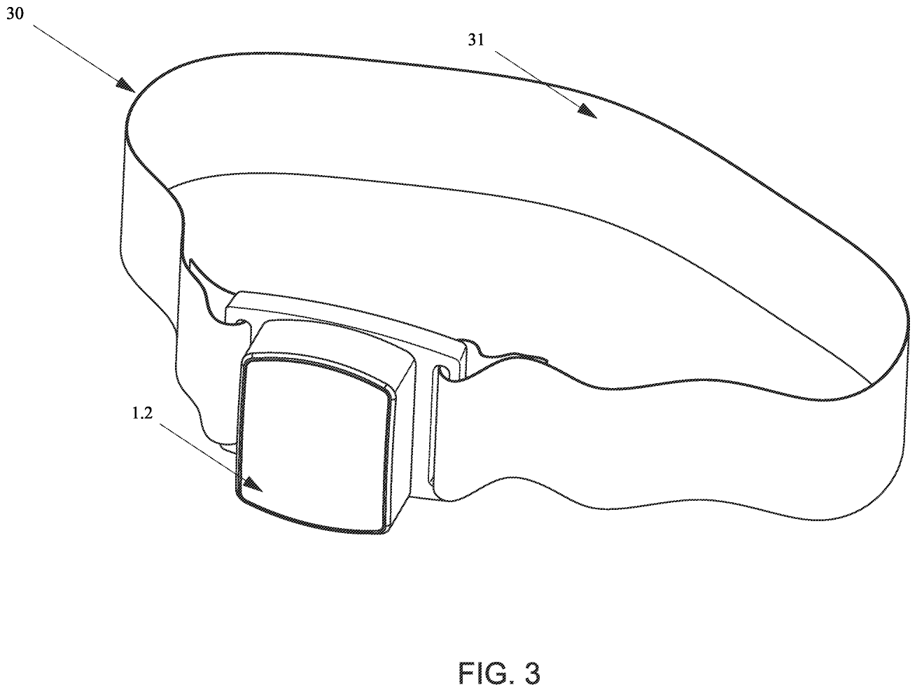

FIG. 3 illustrates a smart belt device 30 including an electronics unit 1.2, in accordance with at least one embodiment of the present disclosure. The smart belt device 30 may include a belt 31 and the electronics unit 1.2. The electronics unit 1.2 may correspond to the electronics unit 1.2 of FIGS. 1A-2. The smart belt device 30 may be used to generate the usage data related to movement of a core portion (e.g., a midsection, stomach, or similar area) of the user. The smart belt device 30 may be attached to a waist, a sternum position, a shoulder, a knee, above an ankle, etc. The smart belt device 30 may be used in combination with the smart ring device 1 of FIGS. 1A-2 to form the smart wearable device.

The belt 31 may include a coupling mount so as to securely fasten the electronics unit 1.2 to the belt 31. The smart belt device 30 may include the belt 31 so as to be ideally placed for tracking both upper and lower body movements of the user whilst being unobstructive and easy to wear and remove.

The belt 31 may include self-adjusting bands of material of varying sizing so as to fit a wide range of users. The materials of the belt 31 may include fabric materials, elastomer materials, or some combination thereof to make the belt 31 size conforming. The belt 31 may provide a secure fit on the appendages of the user. In some embodiments, the belt 31 may include an adjustable belt to fit appendages of different sizes.

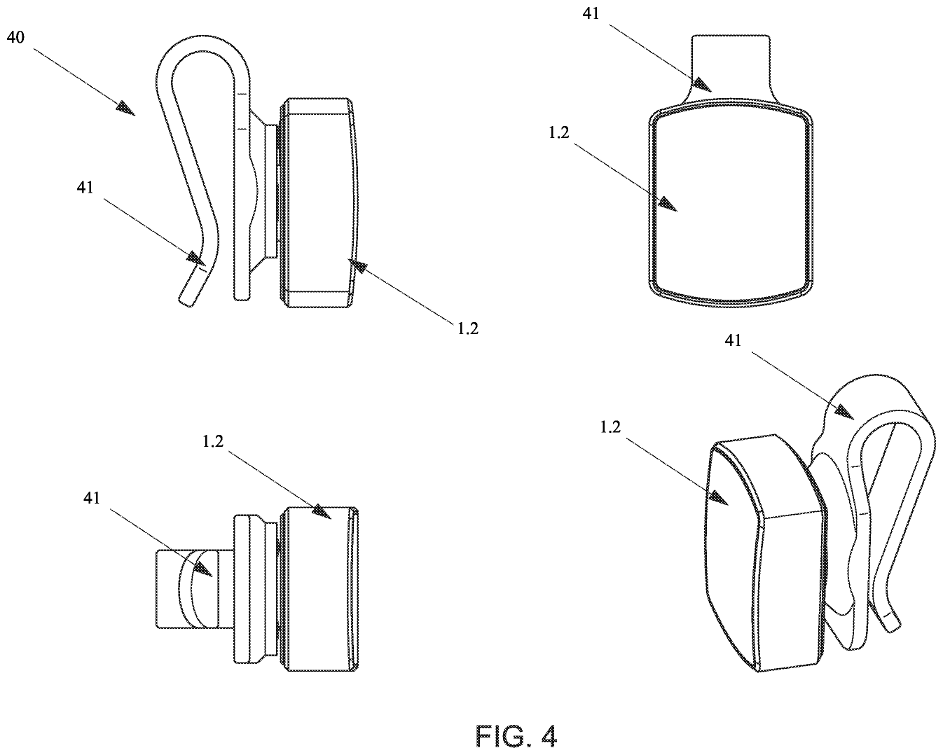

FIG. 4 illustrates a front, a perspective, a side, and a top view of a smart clip device 40, in accordance with at least one embodiment of the present disclosure. The smart clip device 40 may include a clip 41 and the electronics unit 1.2. The electronics unit 1.2 may correspond to the electronics unit 1.2 of FIGS. 1A-3. The smart clip device 40 may be used to generate the usage data related to movement of appendages (e.g., a foot, a calf, a midsection, or similar area) of the user. The smart clip device 40 may be attached to a shoes, shoelaces, socks, or similar article of clothing of the user. The smart clip device 40 may be used in combination with the smart ring device 1 of FIGS. 1A-2, the smart belt device 30 of FIG. 3, or some combination thereof to form the smart wearable device.

The clip 41 may include a coupling mount so as to securely fasten the electronics unit 1.2 to the clip 41. The smart clip device 40 may include the clip 41 so as to be ideally placed for tracking both upper and lower body movements of the user whilst being unobstructive and easy to wear and remove.

The clip 41 may include metal materials, plastic materials, fabric materials, elastomer materials, or some combination thereof to make the clip 41 size conforming to the article of clothing that the smart clip device 40 is attached to.

Terms used herein and especially in the appended claims (e.g., bodies of the appended claims) are generally intended as “open” terms (e.g., the term “including” should be interpreted as “including, but not limited to,” the term “having” should be interpreted as “having at least,” the term “includes” should be interpreted as “includes, but is not limited to,” etc.).

Additionally, if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations.

In addition, even if a specific number of an introduced claim recitation is explicitly recited, it is understood that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” or “one or more of A, B, and C, etc.” is used, in general such a construction is intended to include A alone, B alone, C alone, A and B together, A and C together, B and C together, or A, B, and C together, etc. For example, the use of the term “and/or” is intended to be construed in this manner.

Further, any disjunctive word or phrase presenting two or more alternative terms, whether in the description, claims, or drawings, should be understood to contemplate the possibilities of including one of the terms, either of the terms, or both terms. For example, the phrase “A or B” should be understood to include the possibilities of “A” or “B” or “A and B.”

Additionally, the use of the terms “first,” “second,” “third,” etc., are not necessarily used herein to connote a specific order or number of elements. Generally, the terms “first,” “second,” “third,” etc., are used to distinguish between different elements as generic identifiers. Absence a showing that the terms “first,” “second,” “third,” etc., connote a specific order, these terms should not be understood to connote a specific order. Furthermore, absence a showing that the terms first,” “second,” “third,” etc., connote a specific number of elements, these terms should not be understood to connote a specific number of elements. For example, a first widget may be described as having a first side and a second widget may be described as having a second side. The use of the term “second side” with respect to the second widget may be to distinguish such side of the second widget from the “first side” of the first widget and not to connote that the second widget has two sides.

All examples and conditional language recited herein are intended for pedagogical objects to aid the reader in understanding the invention and the concepts contributed by the inventor to furthering the art, and are to be construed as being without limitation to such specifically recited examples and conditions. Although embodiments of the present disclosure have been described in detail, it should be understood that the various changes, substitutions, and alterations could be made hereto without departing from the spirit and scope of the present disclosure.

What is claimed is:

1. A smart wearable device comprising a smart ring device, the smart ring device comprising:
a ring comprising a coupling mount;
an electronics unit selectively attachable to the coupling mount, the electronics unit configured to generate usage data representative of a movement and a physiological response of a user; and
a feedback system communicatively coupled to the electronics unit, the feedback system configured to display information related to the smart ring device.

2. The smart wearable device of claim 1, wherein the electronics unit further comprising:
a processing unit configured to process the usage data; and determine use of an exercise device by the user; and
a communication system to transmit the usage data to a remote device.

3. The smart wearable device of claim 1, wherein the electronics unit comprises at least one of a motion tracking sensor, a biosensor, and a physiological sensor.

4. The smart wearable device of claim 1, wherein the smart ring device further comprises an electronics enclosure configured to house the electronics unit and the feedback system.

5. The smart wearable device of claim 1, wherein the electronics unit comprises a battery configured to be charged via a magnetic charging port.

6. The smart wearable device of claim 1, wherein the smart ring device comprises a first smart ring device, the smart wearable device further comprising:
a second smart ring device; and
a central smart device, the first smart ring device, the second smart ring device, and the central smart device being configured to generate the usage data representative of bilateral movement of the user.

7. The smart wearable device of claim 1 further comprising a smart belt device, the smart belt device comprising:
a belt comprising a coupling mount;
an electronics unit selectively attachable to the coupling mount of the belt, the electronics unit configured to generate usage data representative of a movement and a physiological response of the user; and
a feedback system communicatively coupled to the electronics unit, the feedback system configured to display information related to the smart belt device.

8. The smart wearable device of claim 1 further comprising a smart clip device, the smart clip device comprising:
a clip comprising a coupling mount;
an electronics unit selectively attachable to the coupling mount of the clip, the electronics unit configured to generate usage data representative of a movement and a physiological response of the user; and
a feedback system communicatively coupled to the electronics unit, the feedback system configured to display information related to the smart clip device.
